(19) United States
(12) Patent Application Publication
(10) Pub. No.: US 2022/0226513 A1
(43) Pub. Date: Jul. 21, 2022

(54) 18F-RADIOLABELED BIOMOLECULES

(71) Applicant: Duke University, Durnam, NC (US)

(72) Inventors: Michael Rod ZALUTSKY, Chapel Hill, NC (US); Ganesan VAIDYANATHAN, Chapel Hill, NC (US); Zhengyuan ZHOU, Durham, NC (US)

(21) Appl. No.: 17/613,715

(22) PCT Filed: May 22, 2020
(86) PCT No.: PCT/US2020/034243

§ 371 Date: Nov. 23, 2021

Related U.S. Application Data
(60) Provisional application No. 62/852,681, filed on May 24, 2019.

(51) Int. Cl. A61K 51/04; A61K 51/10; C07B 59/00; C07D 401/12

(52) U.S. Cl. CPC A61K 51/0453; A61K 51/1093; C07B 2200/05; C07D 401/12; C07B 59/008

(57) ABSTRACT

The application is drawn to ¹⁸F-radiolabeled residualizing agents and biomolecules and methods for radiolabeling biomolecules with radioactive fluorine atoms. The biomolecules have an affinity for particular types of cells and may specifically bind a certain cell, such as a cancer cell. Relevant biomolecules include antibodies, monoclonal antibodies, antibody fragments, peptides, other proteins, nanoparticles and aptamers. The application further provides compositions including such labeled biomolecules, as well as methods of using the labeled biomolecules and/or compositions in imaging applications.

### FIELD OF THE INVENTION

The present invention is drawn to methods of preparing compounds useful for radiolabeling biomolecules and to methods of preparing such radiolabeled biomolecules. The disclosure also provides precursors of radiolabeled biomolecules and the corresponding radiolabeled biomolecules. The compounds can effectively retain radioactivity from biomolecules that become internalized within cells, rendering such compounds useful in the diagnosis of disease, particularly cancer.

### BACKGROUND

A number of monoclonal antibodies (mAbs), mAb fragments and peptides have been labeled with different radionuclides and then used in the detection and treatment of cancers. Many of the most clinically relevant molecular targets such as HER2, epidermal growth factor receptor (EGFR), and the tumor-specific mutant EGERvIII, rapidly internalize into tumor cells. This is a major problem from a labeling perspective because when radiolabeled biomolecules bind to the tumor associated receptors or antigens, they are transported into the cell, get taken up in endosomes/lysosomes where they are degraded rapidly. The difficulty is that these radioactive degradation products can then rapidly escape from the tumor cells. As a result, sufficient radioactivity is no longer present within tumor cells to allow imaging or treatment of the tumor.

As an example, consider labeling a mAb reactive with EGERVIII with radioiodine, in which the standard labeling method used is direct electrophilic substitution. In such cases, as a result of extensive internalization, the radioactivity retained within the tumor is low after receptor binding and subsequent proteolytic degradation. This is due to the rapid washout of the principal catabolite iodotyrosine. To circumvent this problem, “residualizing agents” have been developed which attempt to trap the radioactivity inside the tumor cell after the labeled mAb is internalized. Such residualizing agents for radioiodine include N-succinimidyl 4-guanidinomethyl-3-iodobenzoate (SGMIB); N^{ϵ}-(3-iodobenzoyl)-Lys⁵-N^{α}-maleimido-Gly¹-Geeek, wherein e and k represent residues of D-glutatnic acid and D-lysine, respectively, otherwise known as N²-(2-(2,5-dioxo-2,5-dihydro-1H-pyrrol-1-yl)acetyl)-D-glutamyl-D-glutamyl-D-glutamyl-N′-(3-iodobenzoyl)-D-lysine or IB-MalGeeek; and 2,2′,2″-(10-(2-((6-(3-(((N-succinimidyl)oxy)carbonyl)-5-iodobenzamido)hexyl)amino)-2-oxoethyl)-1,4,7,10-tetraazacyclododecane-1,4, 7-triyl)triacetic acid (SIB-DOTA). Compared with the directly labeled biomolecule, considerable enhancement in tumor retention of radioactivity has been seen when the same biomolecule was labeled with one of these residualizing prosthetic groups.

Positron emission tomography (PET), a state-of-the art imaging technology, is very sensitive and has excellent quantitative capability. The most widely available positron-emitting radionuclide throughout the world is fluorine-18, which has a half-life of 110 min. To combine the advantages of this imaging technique with the targeting properties of internalizing molecules, it is necessary to develop residualizing agents with which these biomolecules can be labeled with fluorine-18. Furthermore, further effective methods for the preparation of such labeled biomolecules are desirable.

### SUMMARY OF THE INVENTION

The invention is drawn to methods, compounds, and compositions for radiolabeling biomolecules (also referred to as macromolecules) with radioactive halogen atoms, and in particular, with ¹⁸F. Advantageously, such methods, compounds, and compositions minimize loss of the radioactive halogen ¹⁸F due to dehalogenation in vivo, preserves the biological activity of the biomolecule, maximizes retention in diseased cells, such as cancer cells, and minimizes the retention of radioactivity in normal tissues after in vivo administration. The biomolecules have an affinity for particular types of cells. That is, the biomolecules may specifically bind a certain cell, such as a cancer cell. Compositions of the invention include the radiolabeled biomolecules. Such biomolecules include antibodies, monoclonal antibodies, antibody fragments, peptides, other proteins, nanoparticles and aptamers. Such examples of biomolecules for purposes of the invention include, diabodies, scFv fragments, DARPins, fibronectin type III-based scaffolds, affibodies, VHH molecules (also known as single domain antibody fragments (sdAb) and nobodies), nucleic acid or protein aptamers, and nanoparticles. Additionally, larger molecules such as proteins >50 kDa including antibodies, monoclonal antibodies, chimeric antibodies, humanized antibodies, and F(ab′)₂ fragments can be used in the methods disclosed herein. In addition, nanoparticles with a size less than 50 nm can be used in the methods disclosed herein. The principles disclosed herein are, in some embodiments, particularly relevant to VHH molecule and other types of small protein constructs, as will be described more thoroughly herein.

The methods of the invention utilize prosthetic compounds that are effective for radiolabeling. As such, the disclosure provides such radiolabeling compounds, as well as precursors to afford such prosthetic compounds. The disclosure further provides radiolabeled macromolecules (e.g., biomolecules) comprising such compounds/radicals and one or more macromolecules. In some such embodiments, these radiolabeled macromolecules are targeted radiotherapeutic agents. The prosthetic compounds and radiolabeled compounds of the invention are useful, e.g., for diagnosing disease.

In one aspect, the disclosure provides a method of preparing an ¹⁸F-labeled biomolecule, comprising: providing a functionalized biomolecule comprising a dienophile; providing a ¹⁸F-containing reagent comprising a diene; and reacting the functionalized biomolecule and the ¹⁸F-containing reagent via an inverse electron-demand Diels-Alder cycloaddition reaction to provide the ¹⁸F-labeled biomolecule. The reagents and resulting ¹⁸F-labeled biomolecule can vary. In one particular, non-limiting embodiment of the method referenced above, the ¹⁸F-containing reagent comprises 6-[¹⁸F]fluoronicotinyl-PEG₄-methyltetrazine. In one particular, non-limiting embodiment, the functionalized biomolecule comprises a biomolecule derivatized with TCO-GK-PEG₄-NHS. The foregoing method can be used to provide, in one specific embodiment, a ¹⁸F-labeled biomolecule of the following formula: [¹⁸F]FN-PEG₄-Tz-TCO-GK-PEG₄-biomolecule, e.g., including, but not limited to, [¹⁸F]FN-PEG₄-Tz-TCO-GK-PEG₄-5F7.

In another aspect of the disclosure is provided a method of preparing an ¹⁸F-labeled biomolecule, comprising: providing a functionalized biomolecule comprising a diene; providing a ¹⁸F-containing reagent comprising a dienophile; reacting the functionalized biomolecule and the ¹⁸-containing reagent via an inverse electron-demand Diels-Alder cycloaddition reaction to provide the ¹⁸F-labeled biomolecule. Again, the reagents and resulting ¹⁸F-labeled biomolecule associated with such a method can vary. In one particular, non-limiting embodiment of the method referenced directly above, the ¹⁸F-containing reagent comprises 6-[¹⁸F]fluoronicotinyl-PEG₄-GK-TCO. In one particular, non-limiting embodiment, the functionalized biomolecule comprises a biomolecule derivatized with —Mal-PEG₄-Tz. The foregoing method can be used to provide, in one specific embodiment, a ¹⁸F-labeled biomolecule of the following formula: [¹⁸F]FN-PEG₄-GK-TCO-Tz-PEG₄-Mal-biomolecule, e.g., including, but not limited to, [¹⁸F]FN-PEG₄-GK-TCO-Tz-PEG₄-Mal 5F7GGC.

In some embodiments of the foregoing methods, the ¹⁸F-containing reagent comprises an [¹⁸F]fluoronicotinyl (FN) group. The dienophile functionality can vary. In some embodiments, the dienophile comprises an octene moiety. For example, one suitable dienophile comprises a trans-cyclooctene (TCO) moiety. Likewise, the diene functionality can vary. In some embodiments, the diene comprises a tetrazine (Tz) moiety. Various other examples of suitable dienes and dienophiles suitable for IEDDAR would be appreciated by one of skill in the art. The ¹⁸F-containing reagent and/or the functionalized biomolecule may, in some embodiments, further comprise a linker. Such linkers can include, for example, PEG and/or renal brush border enzyme-cleavable linkers. The disclosure further provides certain ¹⁸F-labeled biomolecule, comprising a biomolecule conjugated to an ¹⁸F-labeled residualizing agent selected from [¹⁸F]FN-PEG₄-Tz-TCO-GK-PEG₄- and [¹⁸F]FN-PEG₄-GK-TCO-Tz-PEG₄-Mal-, with certain non-limiting examples of such labeled biomolecules of the following formulas: [¹⁸F]FN-PEG₄-Tz-TCO-GK-PEG₄-5F7 and [¹⁸F]FN-PEG₄-GK-TCO-Tz-PEG₄-Mal-5F7GGC. In another aspect of the disclosure is provided a method for the preparation of an ¹⁸F-labeled residualizing agent, comprising: providing a first compound, comprising a guanidine moiety and an alkyne moiety; providing a second compound, comprising a fluoroalkyl azide and a PEG linker; reacting the first and second compounds via click chemistry to give the ¹⁸F-labeled residualizing agent. The click chemistry, in some embodiments, is catalyzed, e.g., by a copper catalyst. The reagents of this method can vary. In one particular, non-limiting embodiment, the first compound is N-succinimidyl 3-42,3-bis(tert-butoxycarbonyl)3uanidine)methyl)-5-ethynylbenzoate and the second compound is 1-azido-2-(2-(2-(2-[¹⁸F]fluoroethoxy)ethoxy)ethoxy)ethane. The disclosure further provides a method for the preparation of an ¹⁸F-labeled biomolecule, comprising: conducting the method referenced immediately above, and reacting the labeling moiety with a biomolecule. The disclosure additionally provides specific ¹⁸F-labeled residualizing agents, e.g., including but not limited to, N-succinimidyl 3-(1-(2-(2-(2-(2-[¹⁸F]fluoroethoxy)ethoxy)ethoxy)ethyl)-1H-1,2,3-triazol-4-yl)-5-(guanidinomethyl)benzoate and corresponding ¹⁸F-labeled biomolecules, comprising a biomolecule conjugated to such ¹⁸F-labeled residualizing agents.

In a still further aspect, the disclosure provides a method of preparing an ¹⁸F-labeled residualizing agent, comprising: providing a boronate precursor comprising a guanidine moiety; and reacting the boronate precursor with a ¹⁸F-fluorodeborylation with an ¹⁸F-containing reagent. The reagents can vary. In some embodiments, the boronate precursor further comprises a TFP ester. In some embodiments, the ¹⁸F-containing reagent is [¹⁸F]tetraethylammonium fluoride. The reacting step can be done, in some embodiments, in the presence of a catalyst, e.g., including, but not limited to, a copper catalyst. Also provided is a method for the preparation of an ¹⁸F-labeled biomolecule, comprising: conducting the immediately foregoing method to provide an ¹⁸F-labeled residualizing agent; and reacting the ¹⁸F-labeled residualizing agent with a biomolecule. The disclosure additionally provides an ¹⁸F-labeled residualizing agent, comprising tetrafluorophenyl 3-[¹⁸F]fluoro-5-guanidinomethylbenzoate, as well as an ¹⁸F-labeled biomolecule, comprising a biomolecule conjugated to such agent.

Furthermore, the disclosure provides a method of imaging cancer cells, comprising employing any one of the ¹⁸F-labeled biomolecules described herein.

Suitable biomolecules labeled according to the methods provided herein and incorporated within the ¹⁸F-labeled biomolecules provided herein can vary widely. In various embodiments, the biomolecule labeled via the foregoing methods is a nanobody. Exemplary nanobodies include, but are not limited to, a HER2-specific nanobody. Specific HER-2-specific nanobodies include, but are not limited to, 5F7, 5F7GCC, 2Rs15d, and variants thereof.

### BRIEF DESCRIPTION OF THE DRAWINGS

In order to provide an understanding of embodiments of the invention, reference is made to the appended drawings, which are not necessarily drawn to scale, and in which reference numerals refer to components of exemplary embodiments of the invention. The drawings are exemplary only, and should not be construed as limiting the invention.

FIG. 1A is a structure of [¹⁸F]AlF-NOTA-PEG₄-Tz-TCO-GK-2Rs15d;

FIG. 1B is an exemplary reaction scheme for the synthesis of [¹⁸F]FN-PEG₄-Tz-TCO-GK-PEG₄-5F7;

FIG. 2A is a structure of [¹⁸F]FN-PEG₄-GK-TCO-Tz-PEG₄-Mal-5F7GGC;

FIG. 2B is a plot of uptake of iso-[¹²⁵I]SGMIB-5F7 (black) and [¹⁸F]FN-PEG₄-GK-TCO-Tz-PEG₄-Mal-5F7GGC (black/white striped) in tumor, kidneys, blood and muscle obtained from a paired-label biodistribution in athymic mice bearing BT474M1 xenografts;

FIG. 2C is a series of maximum intensity projection (MIP) images obtained after administration of [¹⁸F]FN-PEG₄-GK-TCO-Tz-PEG₄-Mal-5F7GGC in a BT474M1 xenograft-bearing mouse:

FIG. 3 is an exemplary reaction scheme for the synthesis of [¹⁸F]RL-III-2Rs15d;

FIG. 4A is a MicroPET/CT image obtained 2 hours after administration of [¹⁸F]RL-III-5F7 in mouse bearing BT474M1BrM3-Fluc intracranial xenografts;

FIG. 4B is a brain section of the mouse in FIG. 2A, stained with H&E;

FIG. 4C is an autoradiography image of an adjacent section of the mouse (where the sizes of the images in FIGS. 4B and 4C are not of the same scale);

FIG. 5A is the structure of SGMIB; and

FIG. 5B is an exemplary reaction scheme for the synthesis of [¹⁸F]TFPFGMB.

### DETAILED DESCRIPTION

The disclosure generally provides certain methods for ¹⁸F labeling of biomolecules, as well as certain precursors and products afforded thereby. The disclosed methods focus largely on the preparation of certain ¹⁸F-labeled residualizing agents and certain exemplary labeled biomolecules; however, such methods can find broader applicability in certain contexts, e.g., in the preparation of other labeled biomolecules as disclosed in U.S. Pat. No. 9,839,704 to Zalutsky et al. or International Patent Application Publication No. WO2018/178936 to Zalutsky et al., which are incorporated herein by reference in their entireties. The disclosure further provides certain ¹⁸F-labeled residualizing agents and certain ¹⁸F-labeled biomolecules, as well as compositions comprising the same, and methods of using such labeled biomolecules and/or compositions for imaging purposes.

Certain abbreviations are used throughout this application, and are used as generally recognized in the art; for convenience, their definitions are as follows.

2Rs15d: a nanobody, as described, e.g., in Vaneycken I, et al (2011) Preclinical screening of anti-HER2 nanobodies for molecular imaging of breast cancer. FASEB J 25:2433-2446, which is incorporated herein by reference in its entirety;

5F7: a nanobody, as described, e.g., in M. Pruszynski et al., Nuclear Medicine and Biology 40 (2013) 52-59;

5F7-GCC: a 5F7 nanobody variant containing a cysteine at its C-terminal, as described, e.g., in M. Pruszynski et al. /Nuclear Medicine and Biology 40 (2013) 52-59;

Boc: tert-butyloxycarbonyl protecting group

DMA: dimethylacetamide

FN: fluoronicotinyl

GK: GlycineLysine

HER-2: Human Epidermal Growth Factor Receptor 2 protein

HPLC: high performance liquid chromatography

IEDDAR: Inverse Electron Demand Diels Alder Cycloaddition Reaction

iso-[¹³¹I]SGMIB: N-succinimidyl 3-guanidinomethyl-5-[¹³¹I]iodobenzoate

Mal: maleimido

PEG: poly(ethylene glycol)

RBBE: renal brush border enzyme

RCY: radiochemical yield

sdAbs: Single domain antibody fragments

[*I]SGMIB: N-succinimidyl 4-guanidinomethyl-3-[*I]iodobenzoate

TCO: Trans-cyclooctene-containing moiety

TFA: Trifluoroacetic acid

TFP: tetrafluorophenyl

Tz: Tetrazine-containing moiety (including, but not limited to, tetrazine, 3-methyl-6-phenyl-1,2,4,5-tetrazine, and further derivatives).

VHH: variable domain of heavy chain-only antibody (aka sdAb, nanobody).

According to one embodiment of the present disclosure, a method of ¹⁸F labeling is provided, which may find particular application in the context of labeling sdAbs and other types of small protein constructs. This method involves an inverse electron-demand Diels-Alder cycloaddition reaction (IEDDAR). Such method involves the steps of: a) providing an ¹⁸F-containing reagent; and b) coupling the ¹⁸F-containing reagent to a functionalized biomolecule (e.g., sdAbs or other small protein construct) using IEDDAR to provide an ¹⁸F-labeled biomolecule.

The disclosed methods can provide for non-site specific labeling or site-specific labeling. Advantageously, such methods can allow for high product yield and retention of affinity and/or immunoreactivity. Generally, the ¹⁸F-containing reagent comprises, in addition to the ¹⁸F moiety, a moiety suitable for IEDDAR (i.e., either a diene or dienophile functionality). Similarly, the functionalized biomolecule comprises, in addition to the biomolecule, the complementary moiety suitable for IEDDAR, such that where the ¹⁸F moiety comprises a diene, the functionalized biomolecule comprises a dienophile, and where the ¹⁸F moiety comprises a dienophile, the functionalized biomolecule comprises a diene. The selection and/or preparation of such reagents, in some embodiments, can provide for either site-specific or non-site specific labeling of the biomolecule.

In one embodiment, the ¹⁸F-containing reagent (which is provided and then coupled to the functionalized biomolecule) comprises, in addition to the label (¹⁸F), a diene suitable for the IEDDAR of step b) above. One exemplary diene is a tetrazine-containing moiety, which can advantageously be included within the ¹⁸F-containing reagent. Advantageously, the ¹⁸F-containing reagent in some such embodiments comprises a fluoronicotinyl moiety, wherein the fluoronicotinyl moiety includes the ¹⁸F. Various other functional groups can be present within the ¹⁸F-containing reagent, so long as such other functional groups do not negatively interfere with the desired IEDDAR. For example, the ¹⁸F-containing reagent may further comprise a linker (e.g., PEG) of varying lengths. One specific exemplary ¹⁸F-containing (diene) reagent that can be effectively utilized in the disclosed method is 6-[¹⁸F]fluoronicotinyl-PEG₄-methyletrazine.

Where the ¹⁸F-containing reagent comprises a diene suitable for the IEDDAR of step b), the functionalized biomolecule employed in this method comprises a dienophile. One exemplary dienophile suitable for the IEDDAR disclosed herein is an octene moiety (e.g., within a TCO functional group). The biomolecule of this “functionalized biomolecule” can generally comprise various sdAbs or other small protein constructs. In one particular embodiment, the biomolecule comprises an anti-HER2 sdAb. One exemplary biomolecule for which this method has been effectively demonstrated is 5F7; however, the method is not limited thereto. The functionalized biomolecule can, in some such embodiments, be further modified with one or more chemical moieties, e.g., one or more linkers. The linker, in certain embodiments, comprises a renal brush border enzyme (RBBE)-cleavable linker. Again, various other functional groups can, in some embodiments, be contained within the functionalized biomolecule, so long as such other functional groups do not negatively interfere with the desired IEDDAR. The functionalized biomolecule, in one particular embodiment useful in the disclosed method, comprises a TCO-GK-PEG₄-NHS linker. Reaction between the functionalized biomolecule (via its dienophile) and the ¹⁸F-containing reagent (via its diene) can provide the desired labeled biomolecule vie IEDDAR.

In other embodiments, the moieties associated with the functionalized biomolecule and the ¹⁸F-containing reagent are switched (e.g., such that the diene is associated with the functionalized biomolecule (rather than with the ¹⁸F-containing reagent as described above) and the dienophile is associated with the ¹⁸F-containing reagent (rather than with the functionalized biomolecule, as described above)).

Advantageously, the ¹⁸F-containing reagent in some embodiments comprises a fluoronicotinyl moiety, wherein the fluoronicotinyl moiety includes the ¹⁸F. In such embodiments, the ¹⁸F-containing reagent (which is provided and then coupled to the functionalized biomolecule) comprises, in addition to the label (¹⁸F), a dienophile suitable for the IEDDAR of step b) above. One exemplary dienophile suitable for the IEDDAR disclosed herein is an octene moiety (e.g., within a TCO functional group). Various other functional groups can be present within the ¹⁸F-containing reagent, so long as such other functional groups do not negatively interfere with the desired IEDDAR. For example, the ¹⁸F-containing reagent may further comprise a linker of varying lengths, wherein the linker comprises, e.g., PEG and/or a renal brush border enzyme (RBBE)-cleavable linker. One specific exemplary ¹⁸F-containing (dienophile) reagent that can be effectively utilized in the disclosed method is 6-[¹⁸F]fluoronicotinyl-PEG₄-GK-TCO.

Where the ¹⁸F-containing reagent comprises a dienophile suitable for the IEDDAR of step b), the functionalized biomolecule employed in this method comprises a diene. One exemplary diene is a tetrazine-containing moiety, which can advantageously be included within the functionalized biomolecule. The biomolecule of this “functionalized biomolecule” can again generally comprise various sdAbs or other small protein constructs. In one particular embodiment, the biomolecule comprises an anti-HER2 sdAb. One exemplary biomolecule for which this method has been effectively demonstrated is 5F7-GGC; however, the method is not limited thereto. Various other functional groups can be present within the functionalized biomolecule, so long as such other functional groups do not negatively interfere with the desired IEDDAR. For example, the functionalized biomolecule may further comprise a linker (e.g., PEG) of varying lengths. In further embodiments, the ¹⁸F-containing reagent comprises a RBBE cleavable linker. One specific exemplary functionalized biomolecule (diene) reagent that can be effectively utilized in the disclosed method is 5F7-GGC-Mal-PEG₄-Tz. Reaction between the functionalized biomolecule (via its diene) and the ¹⁸F-containing reagent (via its dienophile) can provide the desired labeled biomolecule vie IEDDAR.

In one embodiment, the referenced method provides an ¹⁸F-labeled biomolecule (e.g., prepared according to one of the methods referenced herein above). Two exemplary such ¹⁸F-labeled biomolecules are shown below as Formulas I and II.

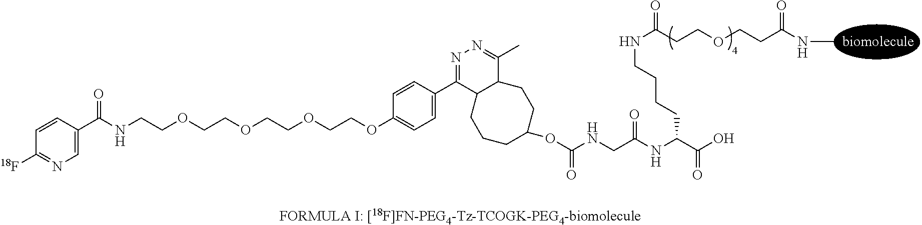

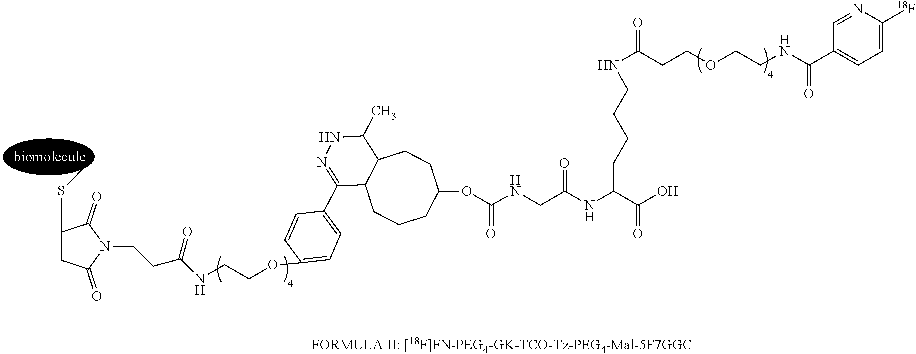

In another embodiment, a method for the preparation of an ¹⁸F-labeled residualizing agent, and an ¹⁸F-labeled biomolecule, comprising a click reaction (e.g., a copper-catalyzed click reaction) is provided. The disclosed reaction comprises the steps of: a) providing a guanidine-bearing compound comprising an alkyne moiety; b) providing a compound comprising a fluoroalkyl azide and including a PEG linker; c) reacting the compounds of steps a) and b) via click chemistry; and optionally, to form the radiolabeled biomolecule, d) reacting the resulting compound with a biomolecule. The copper catalyst employed where the click reaction is copper-catalyzed can vary, and may be any copper-containing compound or salt suitable to catalyze the reaction. In one particular embodiment, the copper catalyst is copper sulfate. Advantageously, the click reaction-based method can, in some embodiments, provide higher radiochemical yields than previously reported for a similar reaction wherein the azide moiety was present on the guanidine-bearing compound, which was reacted with an alkyne (6-[¹⁸F]fluorohexyne). See Glaser, Bioconjugate Chem. 2007, 18, 989-993, which is incorporated herein by reference in its entirety.

The compound comprising a fluoroalkyl azide and including a PEG linker can vary. In one embodiment, this compound is 1-azido-2-(2-(2-(2-[¹⁸F]fluoroethoxy)ethoxy)ethoxy)ethane. Similarly, the compound with which it reacts (i.e., the guanidine-bearing compound) can vary. One exemplary such compound is N-succinimidyl 3-((2,3-bis(tert-butoxycarbonyl)guanidine)methyl)-5-ethynylbenzoate).

The ¹⁸F-labeled residualizing agent provided via steps a)-c) above can be reacted with various biomolecules to afford an ¹⁸F-labeled biomolecule. The biomolecule employed in this method can generally comprise various sdAbs or other small protein constructs (e.g., the types of biomolecules outlined herein above). In one particular embodiment, the biomolecule comprises an anti-HER2 sdAb. Two exemplary biomolecules for which this method has been effectively demonstrated are 2Rs15d and 5F7; however, the method is not limited thereto. This method may provide for simpler synthetic manipulations than previous click chemistry methods for the preparation of such compounds, and can, in some embodiments, provide higher overall radiochemical yields. The disclosure further provides ¹⁸F-labeled biomolecules and intermediates (including ¹⁸F-labeled residualizing agents) afforded by such reactions. One exemplary ¹⁸F-labeled biomolecule is shown in Formula III, below.

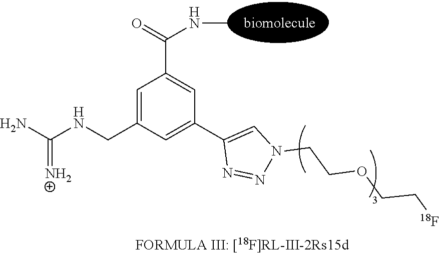

A further method is provided herein for the production of ¹⁸F-labeled residualizing agents and ¹⁸F-labeled biomolecules, which employs fluorodeborylation. In specific embodiments, the method involves providing a boronate precursor containing a TFP ester and a guanidine moiety, wherein the nitrogen atoms on the guanidine moiety are protected (e.g., with Boc groups or other suitable protecting groups that can be introduced/removed under conditions that do not negatively affect the desired reactions). The boronate precursor is subjected to ¹⁸F-fluorodeborylation by treatment with an appropriate ¹⁸F-containing reagent (e.g., [¹⁸F]tetraethylammonium fluoride, [¹⁸F]TEAF). This fluorodeborylation advantageously is catalyzed, e.g., by a copper reagent, including, but not limited to, Cu(Py)₄(OTf)₂. The resulting compound can then be treated to deprotect the nitrogen atoms on the guianidine moiety (e.g., where the protecting group is Boc, these groups can be removed via treatment with TFA).

This deprotected ¹⁸F-labeled residualizing agent can then be conjugated to a biomolecule, which can comprise any of the biomolecules described herein above. In one particular, non-limiting embodiment, the biomolecule is a nanobody, e.g., such as 5F7 or a variant of 5F7. The disclosure further provides ¹⁸F-labeled biomolecules and intermediates afforded by such reactions. One exemplary such ¹⁸F-labeled biomolecule is shown in Formula IV, below.

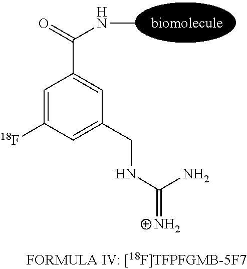 [SMILES: CNC(=O)c1cc([18F])cc(CNC(N)=[NH2+])c1]

The disclosure further provides a composition comprising a radiolabeled biomolecule as disclosed herein (e.g., the ¹⁸F-labeled biomolecule described/shown above, e.g., including those of Formulas I, II, III, and/or IV) in association with a pharmaceutically acceptable adjuvant, diluent or carrier. In a further aspect of the disclosure is provided a method of diagnosing cancer, comprising administering to an individual in need thereof an effective amount of a radiolabeled biomolecule as disclosed herein and/or an effective amount of a pharmaceutical composition as disclosed herein.

The following examples are offered by way of illustration and not by way of limitation.

### EXAMPLE 1

### Fluorine-18 Labeling of an Anti-HER2 sdAb with 6-Fluoronicotinyl Moiety Via the Inverse Electron-Demand Diels-Alder Reaction (IEDDAR) Including a Renal Brush Border Enzyme-Cleavable Linker

### Objectives:

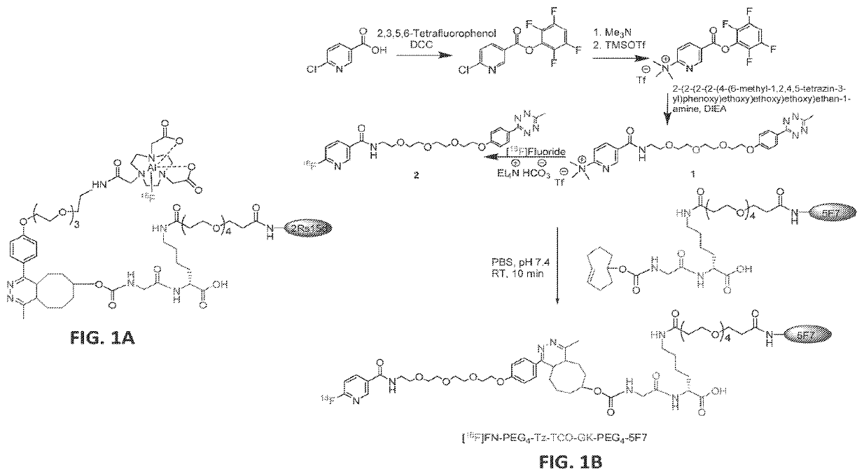

Single domain antibody fragments (sdAbs) are now considered as useful platform for labeling with the short-lived positron emitters such as ¹⁸F due to their low molecular weight, which results in rapid tumor uptake and fast whole-body clearance. However, high levels of renal activity from labeled sdAbs is a significant problem. Previously, we labeled a HER2-specific sdAb, 2Rs15d with ¹⁸F using an [¹⁸F]AlF-NOTA moiety via the tetrazine (Tz)/trans-cyclooctene (TCO) [4+2] inverse electron demand DielsAlder cycloaddition reaction (IEDDAR) with a renal brush border enzyme (BBE)-cleavable linker included in the prosthetic group ([¹⁸F]AlF-NOTA-PEG₄-Tz-TCO-GK-PEG₄-2Rs15d; See FIG. 1A and Zhou et al., Bioconjugate Chem. 2018, 29, 12, 4090-4103, which is incorporated herein by reference in its entirety. While significantly (>15 fold) lower kidney activity levels were achieved for [¹⁸F]AlF-NOTA-PEG₄-Tz-TCO-GK-PEG₄-2Rs15d compared to those for a non BBE-cleavable linker-containing control, tumor uptake was moderate, suggesting that [¹⁸F]-NOTA was not very residualizing. To investigate whether a fluoronicotinyl moiety will result in higher tumor uptake, we modified the above approach by replacing [¹⁸F]AlF-NOTA with the 6-[¹⁸F]fluoronicotinyl (FN) group.

### Methods:

Another HER2-specific sdAb, 5F7, was derivatized with TCO-GK-PEG₄-NHS and then coupled with 6-[¹⁸F]fluoronicotinyl-PEG₄-methyltetrazine ([¹⁸F]2) by IEDAR as shown in FIG. 1B ([¹⁸F]2 was synthesized from N,N,N-trimethyl-5-((2-(2-(2-(2-(4-(6-methyl-1,2,4,5-tetrazin-3-yl)phenoxy)ethoxy)ethoxy)ethoxy) ethyl)carbamoyl)10uanidin-2-aminium triflate (1) (see FIG. 1B). For comparison, 5F7 also was labeled using the validated residualizing agent, N-succinimidyl 3-guanidinomethyl-5-[125I]iodobenzoate (iso-[¹²⁵I]SGMIB; see Choi et al., Nucl. Med. Biol. 2014, 41, 10, 802-812, which is incorporated herein by reference in its entirety). Radiochemical purity (RCP) was determined by SDS-PAGE and immunoreactive fraction (IRF) by the Lindmo method. HER2-binding affinity and paired label (¹⁸F/¹²⁵I) cell uptake assays were performed on HER2-expressing SKOV-3 human ovarian carcinoma cells. Paired label biodistribution was performed in athymic mice bearing SKOV-3 xenografts.

### Results:

The intermediate [¹⁸F]2 was synthesized from precursor 1 in 44.8±3.5% yield. [¹⁸F]FN-PEG₄-Tz-TCO-GK-PEG₄-5F7 was obtained in 76.0% RCY for IEDDAR and its RCP was >99% by SDS-PAGE; K_{D} and IRF were 5.4±0.7 nM and 77.5%, respectively. Uptake of [¹⁸F]FN-PEG₄-Tz-TCO-GK-PEG₄-5F7 in SKOV-3 cells in vitro was 2.4±0.2%, 2.3±0.3%, and 2.6±0.1% of input activity at 1, 2, and 4 h, respectively. Significantly higher values were obtained for co-incubated iso-[¹²⁵I]SGMIB-5F7 (25.9±1.5%, 32.6±2.3%, and 40.8±0.8%). Unlike the in vitro results, SKOV3 xenograft uptake of [¹⁸F]FN-PEG₄-Tz-TCO-GK-PEG₄-5F7 (2.6±2.0% ID/g and 3.7±1.4% ID/g at 1 h and 3 h) was not significantly different from co-injected iso-[¹²⁵I]SGMIB-5F7 {2.0±2.2% ID/g and 6.5±2.6% ID/g, respectively (P>0.05)}. Because of the 7-fold lower levels of ¹⁸F in kidneys, tumor-to-kidney ratios (T:K) for [¹⁸F]FN-PEG₄-Tz-TCO-GK-PEG₄-5F7 were 0.6±0.2 and 4.6±2.0 at 1 h and 3 h, respectively, significantly higher (P<0.05) than those seen for co-injected iso-[¹²⁵I]SGMIB-5F7 (0.1±0.1 and 1.3±1.2). Although the sdAbs are different, the T:K values obtained in this study for [¹⁸F]FN-PEG₄-Tz-TCO-GK-PEG₄-5F7 were considerably higher than those reported before for [¹⁸F]AlF-NOTA-PEG₄-Tz-TCO-GK-PEG₄-2Rs15d (0.4±0.1 and 2.1±0.8 at 1 h and 3 h, respectively; P<0.05 at 3 h).

### Conclusions:

Although the tetrazine moiety is generally considered to be labile for standard ¹⁸F labeling conditions by SNAr, we obtained up to 47% RCY by reducing the amount of base. The sdAb 5F7 modified with a TCO moiety and a brush border enzyme-cleavable linker was labeled with ¹⁸F via IEDDAR using [¹⁸F]2 in excellent yields with retention of affinity and immunoreactivity to HER2. This method of ¹⁸F labeling warrants further investigation for application to sdAbs and other types of small protein constructs.

### EXAMPLE 2

### Fluorine-18 Labeling of an Anti-HER2 sdAb with 6-Fluoronicotinyl Moiety Via the Inverse Electron-Demand Diels-Alder Reaction (IEDDAR) Including a Renal Brush Border Enzyme-Cleavable Linker

### Objectives:

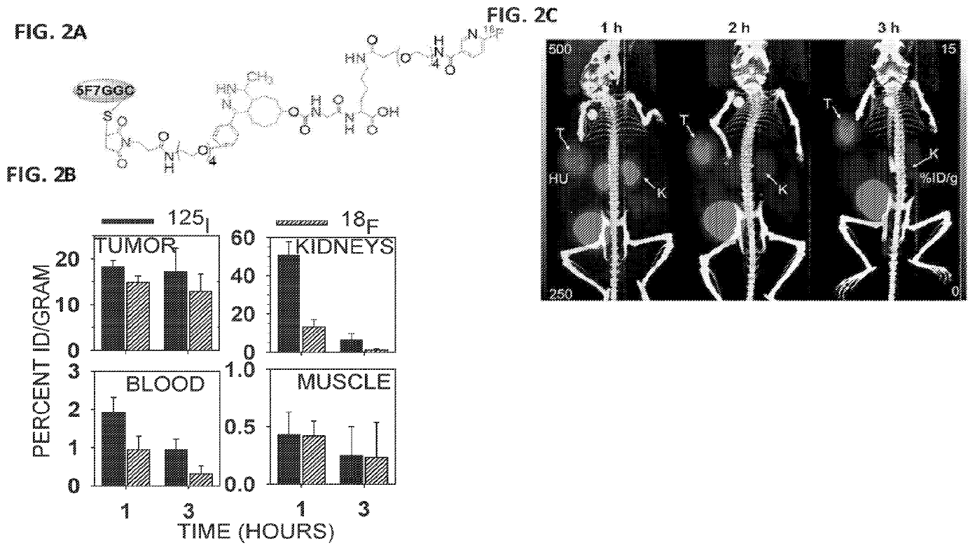

The HER2-specific sdAb, 5F7, was derivatized as follows. First, 5F7-GGC was subjected to Michael addition with Maleimido-PEG₄-Tz, and a 1:1 conjugate of the 5F7-GGC-Mal-PEG₄-Tz was isolated by SE-HPLC. To perform ¹⁸F-labeling using IEDDAR, a ¹⁸F-labeled TCO-containing agent, which also contained a renal brush border enzyme (RBBE)-cleavable linker, a PEG₄ linker, and a 6-[¹⁸F]fluronicotinyl moiety was synthesized ([¹⁸F]FN-PEG₄-GK-TCO). An analogous reagent (precursor) having a trimethylammonium triflate in place of F was also synthesized. The ¹⁸F-labeled agent [¹⁸F]FN-PEG₄-GK-TCO was obtained from the precursor in 47.8±9.4% (n=10) radiochemical yield (RCY). It was conjugated to 5F7-GGC-Mal-PEG₄-Tz in 27.3±8.2% (n=5) yield. The overall decay-corrected yield for the synthesis of [¹⁸F]FN-PEG₄-GK-TCO-Tz-PEG₄-Mal-5F7GGC (FIG. 2A) was 7-8% and the labeled nanobody retained affinity to HER2. Uptake values in tumor, kidneys, blood and muscle from a paired-label biodistribution of [¹⁸F]FN-PEG₄-GK-TCO-Tz-PEG₄-Mal-5F7GGC and iso-[¹²⁵I]SGMIB-5F7 are shown in FIG. 2B. Substantially higher tumor/kidney and tumor/blood ratios for ¹⁸F vs ¹²⁵I were obtained. MIP images in a BT474M1 xenograft-bearing mouse is shown in FIG. 2C. As hypothesized, very little uptake in hepatobiliary organs was seen and a very high contrast image with uptake essentially in only tumor and bladder was seen at 3 h p.i.

### EXAMPLE 3

### Fluorine-18 Labeling of a Single Domain Antibody Fragment with N-Succinimidyl 3(1(2-(2-(2-(2-[¹⁸F]Fluoroethoxy)Ethoxy)Ethoxy)Ethyl)-1H-1,2,3-Triazol-4-Yl)-5 -(Guanidinomethyl)Benzoate, an Alternative Residualizing Prosthetic Agent

### Objectives:

Single domain antibody fragments (sdAbs) are an attractive vector for immunoPET. Earlier, we labeled anti-HER2 sdAbs with ¹⁸F using a residualizing prosthetic agent, N-succinimidyl 3-((4-(4-[¹⁸F]fluorobutyl)-1H-1,2,3 -triazol- 1-yl)methyl)-5 -(guanidinomethyl) benzoate ([¹⁸F]SEBTMGMB or [¹⁸F]RL-I; Vaidyanathan et al., J. Nucl. Med., 2018, 115, 171306, which is incorporated herein by reference in its entirety; and Zhou et al., Mol. Imag. Biol.; 2018. 19, 867-877, which is incorporated herein by reference in its entirety). The prosthetic agent was synthesized by a copper-catalyzed click reaction between an azide-and guanidine-bearing molecule with 6-[¹⁸F]fluorohexyne (FH). However, one drawback of FH is its extreme volatility, making the synthetic manipulations difficult. To overcome this problem, we developed an analogous agent by reversing the click partnersthe guanidine-bearing molecule contained the alkyne moiety, which was clicked with a fluoroalkyl azide that included a PEG linker.

### Methods:

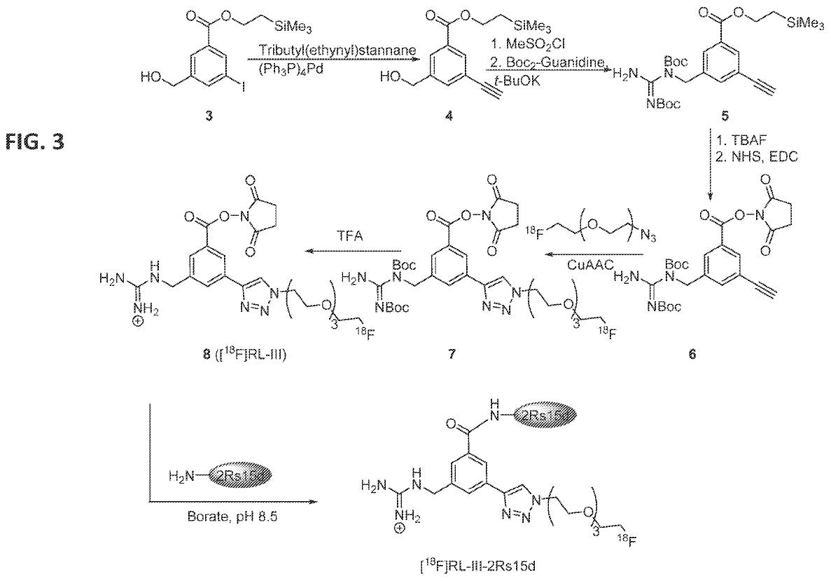

N-succinimidyl 3-((1,2-bis(tert-butoxycarbonyl)guanidino)methyl)-5-ethynylbenzoate (6; FIG. 3) was synthesized in three steps from 2-(trimethylsilyl)ethyl 3-(hydroxymethyl)-5-iodobenzoate (3; see also Choi et al., Nucl. Med. Biol. 2014, 41, 10, 802-812, which is incorporated herein by reference in its entirety). It was clicked with 1-azido-2-(2-(2-(2-[¹⁸F]fluoroethoxy)ethoxy)ethoxy)ethane (see Michel et al., J. Med. Chem. 2011, 54, 4, 939-948, which is incorporated herein by reference in its entirety) and the Boc groups from the resulting intermediate 7 removed to obtain N-succinimidyl 3-(1-(2-(2-(2-(2-[¹⁸F]fluoroethoxy)ethoxy)ethoxy)ethyl)-1H-1,2,3 -triazol-4-yl)-5 -(guanidinomethyl)benzoate (8; [¹⁸F]SFETGMB; [¹⁸F]RL-III; see FIG. 3). An anti-HER2 sdAb 2Rs15d was labeled with ¹⁸F and ¹²⁵I by conjugating it with [¹⁸F]RL-III and N-succinimidyl 4-guanidinomethyl-3 -[¹²⁵I]iodobenzoate ([¹²⁵I]SGMIB; see Vaidyanathan and Zalutsky, Nat. Protocols, 2007, 2, 282-286, which is incorporated herein by reference in its entirety), respectively. The purity of [¹⁸F]RL-III-2Rs15d was evaluated by TCA precipitation, SDS PAGE and size-exclusion HPLC. Its HER2-binding affinity was determined in a saturation binding assay using HER2-expressing BT474M1 human breast carcinoma cells and its immunoreactive fraction (IRF) assessed by the Lindmo method. Paired-label internalization of [¹⁸F]RL-III-2Rs15d and [¹²⁵I]SGMIB-2Rs15d was performed on BT474M1 cells in vitro. The biodistribution of [¹⁸F]RL-III-2Rs15d and [¹²⁵I]SGMIB-2Rs15d were compared in athymic mice bearing subcutaneous HER2-expressing SKOV3 human ovarian carcinoma xenografts.

### Results:

Boc₂-[¹⁸F]SFETGMB was synthesized in an overall radiochemical yield of 22.1±2.4% (n=5) in 125 min. [¹⁸F]RL-III was conjugated to 2Rs15d (2 mg/mL) in 37.5±13.5% yield. Radiochemical purity of [¹⁸F]RL-III-2Rs15d was >96%. K_{d} and IRF were 5.7±0.3 nM and 81.5±1.0%, respectively. The percent of initially bound radioactivity from [¹⁸F]RL-III-2Rs15d that internalized in BT474M1 cells were 10.8±1.0%, 10.6±0.4% and, 9.8±0.7%, respectively, at 1, 2 and 4 h; the corresponding values for [¹²⁵I]SGMIB-2Rs15d were 10.2±0.6%, 10.0±0.4% and, 9.1±0.4%. Uptake in SKOV3 xenografts for [¹⁸F]RL-III-2Rs15d was 4.0±0.5% ID/g, 4.2±1.4% ID/g, and 2.5±0.3% ID/g, at 1, 2 and 3 h, respectively. These values for [¹²⁵I]SGMIB-2Rs15d were 5.5±0.8% ID/g, 6.4±3.1% ID/g and 3.8±0.7% ID/g (P<0.05 except at 2 h). Uptake in a some normal tissues was considerably higher for [¹⁸F]RL-III-2Rs15d compared with [¹²⁵I]SGMIB-2Rs15d (kidney 2-4-fold; liver 25-43-fold; spleen 14-19-fold).

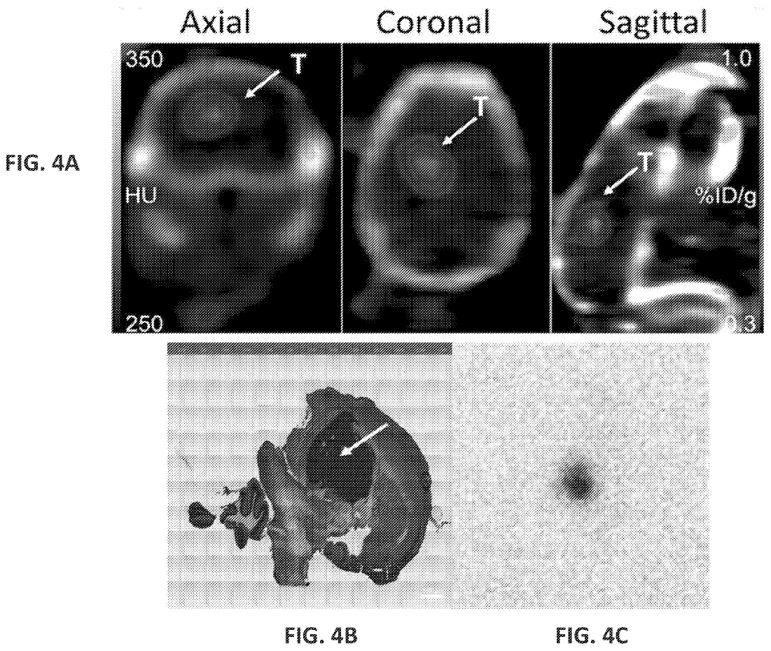

Further, this new residualizing prosthetic agent RL-III was evaluated by labeling two nanobodies (5F7 and 2Rs15d) using [¹⁸F]RL-III. We further evaluated the potential of [¹⁸F]RL-III-5F7 for imaging in mice bearing intracranial tumors. As shown in FIG. 4, the intracranial BT474M1 tumor was clearly visualized with [¹⁸F]RL-III-5F7 (FIG. 4A). Histology and autoradiography of the brain section confirmed the presence and location of tumor (FIGS. 4B&C).

### Conclusions:

The prosthetic agent [¹⁸F]RL-III was synthesized in about 3-fold higher radiochemical yields than that obtained earlier for [¹⁸F]RL-I. The sdAb 2Rs15d was labeled with [¹⁸F]RL-III in similar yields as obtained for [¹⁸F]RL-I giving considerable advantage with respect to RCY for [¹⁸F]RL-III-2Rs15d. Tumor uptake both in vitro and in vivo of [¹⁸F]RL-III-2Rs15d was similar to that for co-incubated/injected [¹²⁵I]SGMIB-2Rs15d demonstrating the residualizing ability of [¹⁸F]RL-III. Normal tissue uptake of [¹⁸F]RL-III-2Rs15d was similar to that seen earlier for [¹⁸F]RL-I-2Rs15d albeit in a different model. These results suggest that [¹⁸F]RL-III is a better prosthetic agent than [¹⁸F]RL-I and warrants further investigation with further structural modifications to reduce uptake of activity from labeled sdAbs in some normal tissues. Use of sdAb labeled using this prosthetic agent in the context of intracranial tumors indicates that [¹⁸F]RL-III-sdAb is a good imaging agent.

### EXAMPLE 4

### Preparation of Tetrafluorophenyl 3-[¹⁸F]fluoro-5-guanidinomethylbenzoate ([¹⁸F]TFPFGMB)

### Objective:

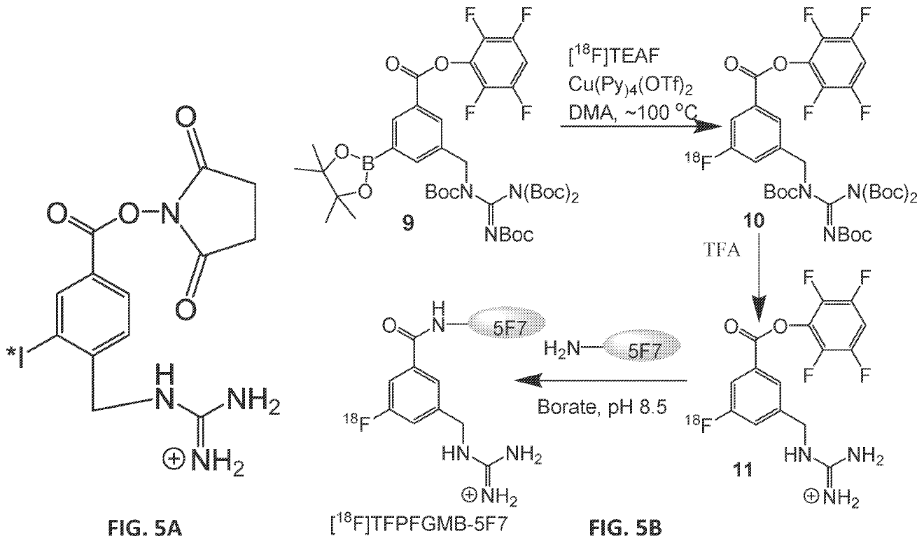

We set out to make an ¹⁸F-labeled residualizing agent similar to SGMIB (FIG. 5A). In particular, we targeted an agent similar to iso-SGMIB, but containing a tetrafluorophenyl (TFP) ester in place of N-hydroxysuccinimide (NHS) ester (([¹⁸F]TFPFGMB; FIG. 5B) in acceptable RCY.

### Methods:

For this, the boronate precursor containing a TFP ester (9, shown in FIG. 5B), wherein all of the nitrogens in the guanidine group was protected with Boc groups, was synthesized and subjected to ¹⁸F-fluorodeborylation by its treatment with [¹⁸F]tetraethylammonium fluoride ([¹⁸F]TEAF), Cu(Py)₄(OTf)₂ in DMA at ˜100° C. for 5-10 min. The product (Intermediate 2 of FIG. 5B) was isolated by normal phase HPLC in 18.0±7.0% (n=4) RCY. The resultant labeled intermediate 10 was deprotected by treatment with TFA. A nanobody variant of 5F7 (“5F7-variant”) was conjugated with Compound 11 (as shown in FIG. 5B) in a yield of 4.7±0.2%; n=2) by incubating a solution of 5F7-variant in borate buffer, pH 8.5 with 3 for 20 min at 37° C.

### Results:

From a single experiment, it was shown that 30.2±1.7%, 40.1±1.5% 45.5±1.6% of input dose was taken up at 1, 2 and 4 h, respectively after HER2-expressing BT474M1 cells were incubated with [¹⁸F]TFPFGMB-5F7-variant at 37° C.; nonspecific binding determined at 2 h was 6.9±0.3%. Activity that was trapped intracellularly at these three time points was 13.8±0.3%, 17.1±1.0% and 24.0±1.4%, respectively.

All publications, patents and patent applications mentioned in the specification are indicative of the level of those skilled in the art to which this invention pertains. All publications, patents and patent applications are herein incorporated by reference to the same extent as if each individual publication, patent or patent application was specifically and individually indicated to be incorporated by reference. Although the foregoing invention has been described in some detail by way of illustration and example for purposes of clarity of understanding, it will be obvious that certain changes and modifications may be practiced within the scope of the embodiments.

What is claimed is:

1. A method of preparing an ¹⁸F-labeled biomolecule, comprising:
providing a functionalized biomolecule comprising a dienophile;
providing a ¹⁸F-containing reagent comprising a diene;
reacting the functionalized biomolecule and the ¹⁸F-containing reagent via an inverse electron-demand Diels-Alder cycloaddition reaction to provide the ¹⁸F-labeled biomolecule.

2. The method of claim 1, wherein the dienophile comprises an octene moiety.

3. The method of claim 1, wherein the dienophile comprises a trans-cyclooctene (TCO) moiety.

4. The method of claim 1, wherein the diene comprises a tetrazine (Tz) moiety.

5. The method of claim 1, wherein the ¹⁸F-containing reagent comprises an [¹⁸F]fluoronicotinyl (FN) group.

6. The method of claim 1, wherein the ¹⁸F-containing reagent comprises 6-[¹⁸F]fluoronicotinyl-PEG₄-methyltetrazine.

7. The method of claim 1, wherein the functionalized biomolecule further comprises a linker.

8. The method of claim 7, wherein the linker comprises a renal brush border enzyme-cleavable linker.

9. The method of claim 1, wherein the functionalized biomolecule comprises a biomolecule derivatized with TCO-GK-PEG₄-NHS.

10. The method of any of claims 1-9, wherein the biomolecule is a nanobody.

11. The method of claim 10, wherein the nanobody is a HER2-specific nanobody.

12. The method of claim 1, wherein the ¹⁸F-labeled biomolecule is [¹⁸F]FN-PEG₄-Tz-TCO-PEG⁻4-GK-biomolecule.

13. A method of preparing an ¹⁸F-labeled biomolecule, comprising:
providing a functionalized biomolecule comprising a diene;
providing a ¹⁸F-containing reagent comprising a dienophile;
reacting the functionalized biomolecule and the ¹⁸F-containing reagent via an inverse electron-demand Diels-Alder cycloaddition reaction to provide the ¹⁸F-labeled biomolecule.

14. The method of claim 13, wherein the dienophile comprises an octene moiety.

15. The method of claim 13, wherein the dienophile comprises a trans-cyclooctene (TCO) moiety.

16. The method of claim 13, wherein the diene comprises a tetrazine (Tz) moiety.

17. The method of claim 13, wherein the ¹⁸F-containing reagent comprises an [¹⁸F]fluoronicotinyl (FN) group.

18. The method of claim 13, wherein the ¹⁸F-containing reagent comprises a renal brush border enzyme-cleavable linker.

19. The method of claim 13, wherein the ¹⁸F-containing reagent comprises 6-[¹⁸F]fluoronicotinyl-PEG₄-GK-TCO.

20. The method of claim 13, wherein the functionalized biomolecule further comprises a linker.

21. The method of claim 20, wherein the linker comprises PEG.

22. The method of claim 13, wherein the functionalized biomolecule comprises a biomolecule derivatized with Mal-PEG₄-Tz.

23. The method of any of claims 13-22, wherein the biomolecule is a nanobody.

24. The method of claim 23, wherein the nanobody is a HER2-specific nanobody.

25. The method of claim 13, wherein the ¹⁸F-labeled biomolecule is [¹⁸F]FN-PEG₄-GK-TCO-Tz-PEG₄-Mal-biomolecule.

26. An ¹⁸F-labeled biomolecule, comprising a biomolecule conjugated to an ¹⁸F-labeled residualizing agent selected from [¹⁸F]FN-PEG₄-Tz-TCO- GK- PEG₄- and [¹⁸F]FN-PEG₄-GK-TCO-Tz-PEG₄-Mal-.

27. The ¹⁸F-labeled biomolecule of claim 26, wherein the biomolecule is a nanobody.

28. The ¹⁸F-labeled biomolecule of claim 27, wherein the nanobody is a HER2-specific nanobody.

29. A method for the preparation of a ¹⁸F-labeled residualizing agent, comprising:
providing a first compound, comprising a guanidine moiety and an alkyne moiety;
providing a second compound, comprising a fluoroalkyl azide and a PEG linker;
reacting the first and second compounds via click chemistry to give the ¹⁸F-labeled residualizing agent.

30. The method of claim 27, wherein the click chemistry is catalyzed by a copper catalyst.

31. The method of claim 27, wherein the first compound is N-succinimidyl 34(2,3-bis(tert-butoxycarbonyl)17uanidine)methyl)-5-ethynylbenzoate and the second compound is 1-azido-2-(2-(2-(2-[¹⁸F]fluoroethoxy)ethoxy)ethoxy)ethane.

32. A method for the preparation of a ¹⁸F-labeled biomolecule, comprising:
conducting the method of any of claims 29-31; and
reacting the ¹⁸F-labeled residualizing agent with a biomolecule.

33. The method of claim 32, wherein the biomolecule is a nanobody.

34. The method of claim 32, wherein the nanobody is a HER2-specific nanobody.

35. An ¹⁸F-labeled residualizing agent, comprising N-succinimidyl 3-(1-(2-(2-(2-(2-[¹⁸F]fluoroethoxy)ethoxy)ethoxy)ethyl)-1H-1,2,3-triazol-4-yl)-5-(guanidinomethyl)benzoate.

36. An ¹⁸F-labeled biomolecule, comprising a biomolecule conjugated to the ¹⁸F-labeled residualizing agent of claim 35.

37. The ¹⁸F-labeled biomolecule of claim 36, wherein the biomolecule is a nanobody.

38. The ¹⁸F-labeled biomolecule of claim 37, wherein the nanobody is a HER2-specific nanobody.

39. A method of preparing an ¹⁸F-labeled residualizing agent, comprising:
providing a boronate precursor comprising a guanidine moiety;
reacting the boronate precursor with a ¹⁸F-fluorodeborylation with an ¹⁸F-containing reagent.

40. The method of claim 39, wherein the boronate precursor further comprises a TFP ester.

41. The method of claim 39, wherein the ¹⁸F-containing reagent is [¹⁸F]tetraethylammonium fluoride.

42. The method of claim 39, wherein the reacting is conducted in the presence of a copper catalyst.

43. A method for the preparation of an ¹⁸F-labeled biomolecule, comprising:
conducting the method of any of claims 39-42; and
reacting the ¹⁸F-labeled biomolecule with a biomolecule.

44. The method of claim 43, wherein the biomolecule is a nanobody.

45. The method of claim 44, wherein the nanobody is a HER2-specific nanobody.

46. A ¹⁸F-labeled residualizing agent, comprising tetrafluorophenyl 3-[¹⁸F]fluoro-5-guanidinomethylbenzoate.

47. A ¹⁸F-labeled biomolecule, comprising a biomolecule conjugated to the ¹⁸F-labeled residualizing agent of claim 46.

48. The ¹⁸F-labeled biomolecule of claim 47, wherein the biomolecule is a nanobody.

49. The ¹⁸F-labeled biomolecule of claim 47, wherein the nanobody is a HER2-specific nanobody.

50. A method of imaging cancer cells, comprising employing the ¹⁸F-labeled biomolecule of any of claim 26-28, 36-38, or 47-49.
